SEQUENCE LISTING (13 sequences)

SEQ ID NO: 1 - DNA, 95 bp, Artificial Sequence
  Synthetic N-aptamer
ttttttgcaatggtacggtactgcgggatgccgcaactggctaattggtgaggctggggcggtcgcgcagcaaaagtgcgcgctacttttgctaa

SEQ ID NO: 2 - DNA, 94 bp, Artificial Sequence
  Synthetic- NV1
  misc_feature (25)..(27): Locked nucleic acid
  misc_feature (40)..(41): Locked nucleic acid
ttttttgcaatggtacggtacttccggatgcggaaactggctaattggtgaggctggggcggtcgtgcagcaaaagtgcacgctactttgctaa

SEQ ID NO: 3 - DNA, 94 bp, Artificial Sequence; identical to SEQ ID NO: 2
  Synthetic- NV2
  misc_feature (82)..(82): Locked nucleic acid
  misc_feature (84)..(84): Locked nucleic acid
  misc_feature (96)..(96): Locked nucleic acid

SEQ ID NO: 4 - DNA, 94 bp, Artificial Sequence; identical to SEQ ID NO: 2
  Synthetic- NV3
  misc_feature (24)..(27): Locked nucleic acid
  misc_feature (40)..(41): Locked nucleic acid
  misc_feature (82)..(82): Locked nucleic acid
  misc_feature (84)..(84): Locked nucleic acid
  misc_feature (96)..(96): Locked nucleic acid

SEQ ID NO: 5 - DNA, 51 bp, Artificial Sequence
  Synthetic
cagcaccgaccttgtgctttgggagtgctggtccaagggcgttaatggaca

SEQ ID NO: 6 - DNA, 51 bp, Artificial Sequence
  Synthetic
caataccgaccttgtgctttgggagtgtcagtccaagggcgccaatggaca

SEQ ID NO: 7 - DNA, 51 bp, Artificial Sequence
  Sythetic
caacaccgaccttgtgctttgggagtgctggtccaagggcgttaatggaca

SEQ ID NO: 8 - DNA, 51 bp, Artificial Sequence
  Synthetic
cagtaccgaccttgtgctttgggagtgctggtccaagggcgttaatggaca

SEQ ID NO: 9 - DNA, 51 bp, Artificial Sequence
  Synthetic
cagcaccgaccttgtgctttgggagtgttggtccaagggcgttaatggaca

SEQ ID NO: 10 - DNA, 51 bp, Artificial Sequence
  Synthetic
cagcaccgaccttgtgctttgggagtgccggtccaagggcgttaatggaca

SEQ ID NO: 11 - DNA, 51 bp, Artificial Sequence
  Synthetic
cagcaccgaccttgtgctttgggagtgctagtccaagggcgttaatggaca

SEQ ID NO: 12 - DNA, 51 bp, Artificial Sequence
  Synthetic
cagcaccgaccttgtgctttgggagtgctggtccaagggcgctaatggaca

SEQ ID NO: 13 - DNA, 51 bp, Artificial Sequence
  Synthetic
cagcaccgaccttgtgctttgggagtgctggtccaagggcgtcaatggaca

(19) United States
(12) Patent Application Publication
(10) Pub. No.: US 2022/0349878 A1
(43) Pub. Date: Nov. 3, 2022

(54) DNA CAPTURE-BASED GRAVITATIONAL FLOW-THROUGH ASSAY FOR ANTIGEN DETECTION

(71) Applicant: University of South Florida, Tampa, FL (US)

(72) Inventors: Subhra MOHAPATRA, Lutz, FL (US); Shyam MOHAPATRA, Lutz, FL (US); Ryan GREEN, Tampa, FL (US)

(21) Appl. No.: 17/733,265

(22) Filed: Apr. 29, 2022

Related U.S. Application Data
(60) Provisional application No. 63/201,469, filed on Apr. 30, 2021.

(51) Int. Cl. G01N 33/53; G01N 33/543; B01L 3/00

(52) U.S. Cl. CPC G01N 33/5308; G01N 33/54386; G01N 33/54393; B01L 3/5023; B01L 2300/069

(57) ABSTRACT

The present disclosure provides novel gravitational flow assays for detecting an antigen in a biological sample using DNA-capture sequences. Specifically, gravitational flow assays and methods of detecting viruses, including coronaviruses, are provided.

### CROSS-REFERENCE TO RELATED APPLICATIONS

The present application claims priority to U.S. Provisional Patent Application No. 63/201,469 that was filed Apr. 30, 2021, the entire contents of which are hereby incorporated by reference.

### REFERENCE TO A SEQUENCE LISTING

This application is being filed electronically via EFS-Web and includes an electronically submitted Sequence Listing in .txt format. The .txt file contains a sequence listing entitled “173738_02395_ST25.txt” created on Apr. 29, 2022 and is 3,822 bytes in size. The Sequence Listing contained in this .txt file is part of the specification and is hereby incorporated by reference herein in its entirety.

### FIELD OF THE INVENTION

The disclosed technology is generally directed to test kits for ultra-rapid detection and diagnosis of an infectious agent

### BACKGROUND OF THE INVENTION

Many diseases are first diagnosed using screening tests for antigens and are confirmed by additional testing. It is known that screening tests must possess a high degree of sensitivity, whereas confirmatory assays must possess a high degree of specificity. Tests with high sensitivity are known to produce few false-negative results, whereas tests with high specificity produce few false-positive results. It is difficult to produce a test kit having both high sensitivity and a high degree of specificity. Those knowledgeable in the field recognize that a single kit for use in a field, home environment, or a doctor's office cannot meet both sensitivity and specificity in a rapid assay for disease causing antigen(s) using their corresponding antibodies.

A case in point is an infectious disease named Corona Virus Disease (COVID-19) caused by Severe Acute Respiratory Syndrome Corona Virus 2 (SARS-CoV-2) which was identified in Wuhan, China in December 2019, and resulted in an ongoing pandemic [1, 2]. As of Oct. 10, 2021, more than 237.8 million (>44 MM in USA) people have been infected resulting in >4.8 million (>709,000 in USA) deaths dues to COVID-19 across 218 countries and territories around the globe as per the data of the World Health Organization (WHO). COVID-19 is caused when a person breathes or swallows viral particles from an infected person. It can be also caused by touching mouth, nose, or possibly eyes after touching with an object or surface with the virus on it [3, 4]. COVID-19 spread rapidly worldwide and causes most infected individuals to have flu-like symptoms and thus lose the ability to identify infected individuals without a confirmatory SARS-CoV-2 test [5]. To avoid more deaths, there is an urgent need to contain the pandemic which is why it is very important to quickly identify infectious individuals and control the spread of new infections [5, 6].

The current gold standard to detect SARS-CoV-2 is a quantitative polymerase chain reaction (qPCR) based assay from a nasal or pharyngeal swab or sputum which has several potential error generating steps, takes several hours for sampling and evaluation, and requires special reagents, equipment and trained laboratory scientists [5-7]. Lately, new qPCR-based reagents, such as LAMP PCR techniques to detect SARS-CoV-2 have emerged yet with the extraordinary pricing and supply chain issues, it is still not possible to scale up to millions of tests per day. Testing for viral genetic material is possible using advanced PCR and reverse transcription PCR techniques, but these techniques are not appropriate for a rapid test kit for use at point of care. Instead, these techniques are expensive and time consuming. Advances in PCR on a chip and the like offer some promise for reducing costs and allowing point of care PCR methods, but practical, commercial devices using these techniques remain elusive. Regardless, widespread, routine screening using a PCR-based detection method is impractical.

On the contrary, rapid antigen tests are cheaper with faster results and may be possible to scale testing to more than required, which would reduce the burden on the laboratories, control the spread and facilitate safe re-opening of the businesses and the community. Most of the rapid antigen tests are developed upon the Lateral Flow Assay (LFA) platform as they are easy to use, cheap and flexible. Lateral flow tests, also called immunochromatographic strip tests, are used for specific screening or semi-quantitative detection of many analytes including antigens and antibodies. Samples may either be used alone or with an extraction reagent, or running buffer, which is then placed on a sample pad on one end of a test strip. The test strip also includes amembrane. A signal reagent is solubilized and binds to an antigen if present in the sample and moves through the membrane by capillary action. The complex is then captured by a second antibody, which produces a visible line, indicating presence of the antigen.

Lateral flow tests are slow, but contrast is improved between the visible line and the background compared to directly depositing the sample in the test area. For this reason, lateral flow tests dominate the market for enzymatic testing of bodily fluids. For example, lateral tests are known that can use nasopharyngeal, nasal or blood samples. A major drawback with the LFA platform is that they are affected by mass transport limitations and binding kinetics which means the target antigen needs to bind to the analyte and then be carried across the membrane causing “Hook Effect”, which could affect the speed and sensitivity of the test if the concentration of antibody or analyte is too high [10, 11]. There are also some commercial and manufacturing limitations for LFA such as pretreatment and assembly of multiple components such as sample pad, conjugate pad, nitrocellulose membrane, and absorbing pads which can cross-react and give false-positive results [12]. Recently, some antigen tests have received an Emergency Use Authorization from the FDA. Lateral flow-thru assays take ˜15 mins to give results and have lower sensitivity compared to vertical flow.

Direct, flow-through test kits are known to be rapid but are seldom used in practice due to the complexity of the protocol required to provide enough contrast between the indicator and the background membrane. Within the field, there was a general acceptance that lack of contrast makes flow through test kits less sensitive than lateral flow test kits, and this taught away from the use of flow through test kits. Also, it was thought that complicated procedures and instructions were necessary for washing and rewashing the kits making results, in practice, less consistent than results for a lateral flow test kit, which also mitigates against flow-through tests.

Mahajan, in US Patent Publication No. 2004/0023210, discloses a diagnostic kit for detection of antibodies of Hepatitis C virus in human serum and plasma, which comprises a base, an immunofiltration membrane of nitrocellulose mounted over an absorbent pad disposed on the base, and a top cover removably attached to the base having a central hole conforming to the membrane's circumference. Antigens such as NS3, NS4, and NS5 are immobilized on the membrane and visualized with a Protein A conjugate. This reference teaches that the pore size of the nitrocellulose membrane is 0.8-1.5 microns. The pore size is poorly correlated with specificity and sensitivity.

Chu, in U.S. Pat. No. 5,885,526, discloses a flow-through test device having a reaction membrane that includes porous material, such as nitrocellulose. A small pore size is thought to be needed when using nitrocellulose membranes to provide a greater area for “immobilizing receptor molecules”. Chu teaches that larger pore sizes lead to decreased assay sensitivity, as described in col. 5, Ins. 53-56. The examples in Chu also teach away from increasing flow rate, which Chu describes as decreasing interaction time between a target molecule in the sample and an immobilized receptor on the reaction membrane. Thus, assay sensitivity decreases as disclosed in col. 5, Ins. 57-60. Again, pore size is a poor predictor of sensitivity and specificity.

There is a need for new, fast and sensitive assays for point of care applications, and are readily made, transported and stored for ease of use.

### BRIEF SUMMARY OF THE INVENTION

The present disclosure provides methods and kits for detecting an antigen in a biological sample by gravitational flow.

In one aspect, the disclosure provides a direct gravitational flow through assay for detecting an antigen in a sample comprising: (a) a cassette comprising an absorbent pad; (b) a reaction matrix layered on top of the absorbent pad within the cassette, the reaction matrix comprising nitrocellulose backed with cellulose or polymer membrane, and (c) a detection agent comprising (i) an antigen-specific DNA capture sequence (DCS) linked to the matrix and (ii) a second DCS linked detection reagent; wherein when the assay is contacted with a biological sample capable of gravitational flow through the matrix and absorbent pad, and wherein the formation of a complex between the detection reagent and sample, DCS and an antigen encoding sequence on the matrix within the cassette detects the presence of the antigen within the assay.

In another aspect, the disclosure provides a method of detecting an antigen in a sample, the method comprising: a) contacting a sample with a reaction matrix within a gravitational flow through assay described herein, b) allowing the sample to flow through the reaction matrix by gravity; c) contacting the reaction matrix with a solution comprising a second detectable agent; and d) detecting the presence of a complex of the antigen-specific DCS, detection tag, second detectable agent and a nucleic acid sequence from the sample, wherein detection of the complex indicates the presence of antigen in the sample.

In another aspect, the disclosure provides a method of detecting coronavirus in a sample, the method comprising: a) contacting a sample with a reaction matrix within a gravitational flow through assay, the gravitational flow through assay comprising: (i) a cassette comprising an absorbent pad; (ii) a reaction matrix comprising nitrocellulose layered on top of the absorbent pad within the cassette, the reaction matrix complexed with a coronavirus-specific DNA capture sequence (DCS); b) allowing the sample to flow through the reaction matrix by gravity; and c) detecting the presence of a complex of the coronavirus-specific DCS and a coronavirus nucleic acid sequence from the sample, wherein detection of the complex indicates the presence of coronavirus in the sample.

In another aspect, the disclosure provides a method of detecting coronavirus in a sample, the method comprising: a) contacting a sample with a reaction matrix within a gravitational flow through assay, the gravitational flow through assay comprising: (i) a cassette comprising an absorbent pad; (ii) a reaction matrix comprising nitrocellulose layered on top of the absorbent pad within the cassette, the reaction matrix complexed with a coronavirus-specific DNA capture sequence (DCS) linked to a detection tag; and b) allowing the sample to flow through the reaction matrix by gravity; c) contacting the reaction matrix with a solution comprising a second detectable agent; d) washing the reaction matrix with a solution; and e) detecting the presence of a complex of the coronavirus-specific DCS, detection tag, second detectable agent and a nucleic acid sequence from the sample, wherein detection of the complex indicates the presence of coronavirus in the sample.

In a further aspect, the disclosure provides a method of making a gravity flow through assay for coronavirus, comprising: a) conjugating a coronavirus-specific DNA capture sequence (DCS) with a detection tag; b) contacting the coronavirus-specific DCS with a reaction matrix comprising nitrocellulose; c) exposing the reaction matrix to UV light for a sufficient time to link the coronavirus-specific DCS with the reaction matrix; d) layering in a cassette (i) an absorbent pad, (ii) the reaction matrix comprising the DCS linked with a detection tag.

In yet another aspect, the disclosure provides a kit comprising a gravity flow through assay for coronavirus comprising (a) an assay comprising (i) a cassette containing an absorbent pad; (ii) a porous reaction matrix layered on top of the absorbent pad within the cassette, the reaction matrix comprising nitrocellulose; and (iii) a detection agent comprising (i) a coronavirus-specific DNA capture sequence (DCS) linked to the matrix; (b) a solution comprising a detectable molecule; (c) a wash solution and (d) instructions for use.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention.

FIG. 1: Assembly of the gravitational flow-through device. Panel A shows components of a general assembly required for assay. A single well device, which is used for single (test) or double reaction (a negative and positive) is schematically depicted. Panel B shows three different prototypes are shown including single well, two well, and two well and a swab devices.

FIG. 2. Design of LNA incorporating DCS for N antigen. Panel A shows each DCS has 2 stem regions that were used for LNA substitution. The nucleotide numbers are as follows: N1=17-21/26-30, N2=60-63/73-76. Panel B shows the sequences of LNA-substituted DCS are shown. Plus (+) indicates that next base is LNA. Sequences in panel B are SEQ ID NOs: 1-4, listed top to bottom. Panel C shows binding assay on LNA substituted DCS s for N antigen. Test was conducted in a 2 well GFT cassette with one well for negative control (C) and one for test (T). All tests were conducted with DCS at 250 uM and with MgCl2 either at 1 mM or at 5 mM. The control N was conducted with 5 mM MgCl2.

FIG. 3: Panel A shows N-DNA capture tested on Matrix 1, i.e., FT060 membrane in duplicate using 5×LOD virus sample. Panel B shows ImageJ quantification of images from Panel A.

FIG. 4: Panel A shows predicted secondary structure of the DNA capture sequences used targeting the Spike (S) antigen of SARS-CoV-2 virus. Panel B shows the predicted interaction of DCS sequences with SARS COV-2 spike protein RBD residues (SEQ ID NOs: 5-13 top to bottom). Panel C shows DNA capture sequences with substitution of nucleotides that contact the residues.

FIG. 5: Panel A shows S-DNA capture tested on Matrix 1, i.e., FT060 membrane in duplicate using 5×LOD virus sample. Panel B shows ImageJ quantification of images from A.

FIG. 6: Test using NV3-biotin+Streptavidin-gold and varying membrane types, 5×LOD virus sample.

FIG. 7: Detection of SARS CoV-2 N, S, N+S antigens on Matrix 2 with or without amine S linking.

FIG. 8: Determining Stability of the NV3 DNA Capture Sequences.

FIG. 9A: Shows testing of the N antigen-DNA capture sequences (DCS) for detection of SARS-CoV-2 N by linking DCS with gold nanoparticles. Panel 9A shows synthesis of gold nanoparticles.

FIG. 9B: Shows testing of the N antigen-DNA capture sequences (DCS) for detection of SARS-CoV-2 N by linking DCS with gold nanoparticles. Panel 9B shows the size of the gold nanoparticles.

FIG. 9C: Shows testing of the N antigen-DNA capture sequences (DCS) for detection of SARS-CoV-2 N by linking DCS with gold nanoparticles. Panel 9C shows the size of the gold nanoparticles.

FIG. 9D: Shows testing of the N antigen-DNA capture sequences (DCS) for detection of SARS-CoV-2 N by linking DCS with gold nanoparticles. Panel 9D shows Thiolated gold conjugated with the DCS.

FIG. 9E: Shows testing of the N antigen-DNA capture sequences (DCS) for detection of SARS-CoV-2 N by linking DCS with gold nanoparticles. Panel 9E shows comparison of unconjugated and conjugated gold NPs using 3-step protocol using 3×LOD inactivated virus and No Virus cassette was used as control

FIG. 9F: Shows testing of the N antigen-DNA capture sequences (DCS) for detection of SARS-CoV-2 N by linking DCS with gold nanoparticles. Panel 9F shows comparison of unconjugated and conjugated gold NPs using 3-step protocol using 3×LOD inactivated virus and No Virus cassette was used as control

FIG. 9G: Shows testing of the N antigen-DNA capture sequences (DCS) for detection of SARS-CoV-2 N by linking DCS with gold nanoparticles. Panel 9G shows a comparison of NV3 with scrambled NV3 for a nasal swab spiked with 3×LOD inactivated virus.

FIG. 10: Panel A shows stability of gold NPs reagents stored at RT for 45 days and Panel B shows stability of gold NPs reagents stored at RT for 77 days.

FIG. 11: Determining Limit of detection of the test using Matrix 2. Panel A shows results from triplicate trials of concentrations ranging from 5,000 TCID₅₀/mL to 156.25 TCID₅₀/mL. Panel B shows the limit of detection (LOD).

FIG. 12: An initial cross-reactivity study was performed using 54, samples of 1×10⁵ TCID50/mL common respiratory viruses on amine S-linked Matrix-2 and the 3-step gold conjugated NV3 detection method. No cross-reactivity was detected with the viruses tested.

FIG. 13: Demonstrates detection of all variants using OMC-01 test using as-linked cassettes at 3×LOD.

FIG. 14: Detection of SARS CoV-2 N protein in varying dilutions of blood that has been spiked with 5×LOD CoV-2 virus. FIG. 15: Demonstrates detection of SARS CoV-2 N protein in urine in a test using 5 uL sample, 2 uL NV3 or Sc conjugated AuNP. Panel A shows Urine vs PBS at 3×LOD in a test Urine sample #1 showing no difference. Panel B shows test results in Urine sample #2 at 3, 5, 10×LOD CoV-2 virus.

### DETAILED DESCRIPTION OF THE INVENTION

Disclosed herein is a direct gravitational flow through assay and kit for detecting an antigen, more specifically infectious agents, such as coronavirus. The assay and kit described herein allow for ultra-rapid detection of an antigen (e.g., coronavirus) using a captured DNA-based method. The assay is membrane-based where fluids are applied vertically instead of in parallel, as done in lateral flow assays. This work addresses the limitations of lateral flow assays (LFA) by combining rapidity and simplicity of direct flow-through tests with the specific affinity of aptamer-based antigen detection. To avoid batch-to-batch variation in antibody preparation and to side-step reduced assay sensitivity due to continued mutations of coronaviruses, the assay utilizes aptamers in a non-antibody-based approach to detect antigens. The altered aptamer sequences, known as DNA Capture Sequences (DCS) were developed for high affinity and antigen specificity. Broadly, the assay is comprised of a liquid-impervious cassette holding an absorbent pad layered on top with a reaction matrix. The reaction matrix is comprised of covalently linked DCS and a detection molecule. A liquid biological sample may be introduced to the reaction matrix and drawn through the reaction matrix by the absorbent pad. The assay is read out by the presence or absence of a complex of the detection molecule, DCS, and an antigen (e.g., coronavirus) on the reaction matrix.

Lateral flow assays and other assays previously used rely on antigen detection using antibodies and have several limitations, including antibody preparations have batch to batch variation and the ability of viruses to mutate creating new mutants and new variants resulting in reduction in sensitivity. The present disclosure uses a non-antibody-based approach to detect SARS-COV-2 antigens. Rapid SARS-CoV-2 antigen tests are based on the principle of detecting specific coronavirus protein or protein fragments for very quick results without special reagents and equipment minimizing erroneous steps. Specific antigens can be targeted and detected by using a short single-stranded oligonucleotides (ssDNA), also known as aptamers, which when folded into tertiary structures bind to the target with high affinity and specificity. The present disclosure assay altered the aptamer sequences to provide high-affinity and specific antigen recognition and increase stability of the oligonucleotides and hence coined the term DNA Capture Sequences (DCS) to describe these oligonucleotides.

Further, to address the limitations of the current LFA, the present disclosure provides a direct gravitational Flow Through (GFT) Assay, which is also a membrane based assay, however, fluids are applied to the surface membrane vertically instead of orthogonally. GFT assay has a huge advantage over LFA due to its simplicity of use, cost effectiveness, fast response time, robustness, and it also has fewer components. The present disclosure combines the method detecting SARS-CoV-2 virus by combining the Aptamer-based approach using a GFT system. Because the test takes 1 minute for the detection after the addition of developer and test takes about two minutes from start to finish, this ultra-rapid test was designated as One Minute CoviTest (OMC-01) test for the purpose of the clinical studies.

In one embodiment, and as described in the Examples, the SARS CoV-2 GFT assay was developed using two different compatible membranes (FIG. 1). Either Matrix-1 one, which is a paper backed nitrocellulose membrane 600 μm thick with a pore size of 0.6 μm or Matrix-2, which is a polyester supported nitrocellulose membrane with pore size 0.45 μm can be used. Aptamers with a specific affinity to bind the SARS CoV-2 spike protein are covalently attached to either Matrix-1 or Matrix-2 by the application of UV light. The assay is performed in either a 3-step or 4-step procedure. The 4-step procedure begins with depositing the liquid sample of nasal or nasopharyngeal origin onto the cassette containing either Matrix-1 or Matrix-2. Then, DCS conjugated to biotin is added to the membrane followed by a colloidal suspension of 10 OD streptavidin conjugated 15 nm gold nanoparticles. Finally, a wash buffer is added to allow unbound nanoparticles to flow through the membrane and the result is observed. In the 3-step procedure the sample is applied to the matrix, a detection reagent consisting of DCS covalently linked to gold nanoparticles is added and unbound DCS is washed away revealing the result.

In one embodiment, the disclosure provides a direct gravitational flow through assay for detecting an antigen in a sample comprising: (a) a cassette comprising an absorbent pad; (b) a reaction matrix layered on top of the absorbent pad within the cassette, the reaction matrix comprising nitrocellulose backed with cellulose or polymer membrane, and (c) a detection agent comprising (i) an antigen-specific DNA capture sequence (DCS) linked to the matrix and (ii) a second DCS linked detection reagent; wherein when the assay is contacted with a biological sample capable of gravitational flow through the matrix and absorbent pad, and wherein the formation of a complex between the detection reagent and antigen within the sample, DCS and an antigen encoding sequence on the matrix within the cassette detects the presence of the antigen within the assay.

The term “gravitational flow through assay” refers to gravity acts to move the biological sample contacted with the detection matrix within the cassette in the direction of gravity through the reaction matrix and pad (as opposed to laterally to the surface as in lateral flow) in order to contact the antigens within the sample with the detection agents within the reaction matrix.

The assay described herein may be used to detect and diagnose target antigens in a biological sample. The term “antigen” or “target antigen” as used herein refers to a foreign molecule or molecular structure of any foreign particulate matter. Particularly, the antigen may in some instances, specifically bind to an antibody or T-cell receptor and elicit an immune response, however, for the purposes of the present technology, the antigen only needs to provide evidence of the presence of the foreign molecule within the subject. Suitable target antigens include infectious agents, for example, viruses, bacteria, fungi, among others. In a preferred embodiment, the antigen is a viral antigen, for example, an antigen to SARS-COV-2. In some embodiments, the antigen is a polynucleotide associated with the infectious agent, e.g., DNA or RNA associated with the virus, bacteria or fungus.

The term biological sample refers to samples derived from a subject, for example, a material from nasal or nasopharyngeal origin, saliva, blood (e.g., arteriolar, venous, or capillary) , urine, mucus, semen, interstitial fluid, blood plasma, pus, bile serum, cerebrospinal fluid, synovial fluid, peritoneal fluid, pleural fluid, amniotic fluid, lymphatic fluid, or other biological materials that may contain antigens. In one embodiment the biological sample is a liquid. In another embodiment, a nonfluid biological sample may be processed into a liquid sample with an inert carrier solution, such as an aqueous buffer solution, to create a liquid sample. In another embodiment a biological sample may be diluted in an inert solution to be used for the antigen detection.

The absorbent pad and reaction matrix may be housed in a cassette. The term cassette refers to a liquid-impervious case with a central hole exposing the reaction matrix to which a sample may be contacted. The role of the cassette is to both support the absorbent pad and maintain uniform contact between the absorbent pad and the reaction matrix. In one preferred embodiment the cassette is made of a polymer or plastic, however other suitable materials are contemplated (e.g, glass, polymer coated cardboard, etc.).

The cassette comprises a reaction matrix layered on top of an absorbent pad. The reaction matrix comprises nitrocellulose backed with a cellulose or polyester membrane and is capable of being linked or complexed to the detection agent.

In one preferred embodiment the reaction matrix includes paper-backed or cellulose backed nitrocellulose. In one embodiment, the paper-back nitrocellulose is about 100 μm to about 1000 μm thick. In one preferred embodiment, the nitrocellulose is about 600 μm thick. In some embodiments the pore size of the nitrocellulose is about 0.1 μm to about 1.2 μm in diameter, alternatively about 0.4 μm to about 0.8 μm diameter. In one preferred embodiment the pore size is 0.6 μm. Another preferred embodiment includes the reaction matrix comprising a polyester-supported nitrocellulose membrane. In some embodiments, the pore size of the polyester-supported nitrocellulose is about 0.1 μm to about 1 μm. In one preferred embodiment the pore size is 0.4 μm to about 0.8 μm. In a preferred embodiment, the pore size is about 0.4 μm to about 0.5 μm, preferably about 0.45 μm. The pore size may vary depending upon the nature of antigen being detected. The most desirable thickness is in the range of 100-800 μm.

The assay is additionally comprised of an absorbent pad positioned beneath the reaction matrix. The absorbent pad may be optimized to completely absorb and retain the mixture of fluids that has flowed through the reaction matrix, e.g., the biological sample, the solution of DCS, and the solution of detection molecule, and optionally, if used, the wash buffer. Any conventionally employed absorbent material that is capable of wicking or drawing liquid through a porous membrane, such as, for example, by capillary action may be used. In some embodiments the absorbent pad may be made of cellulose paper, cellulose acetate fibers, polyester, polyolefin, or other such materials. In some embodiments the absorbent pad is comprised of layers. Layers of commercially available filter paper or bathroom tissue can be also used. The absorbent body provides a means to collect the sample by providing uniform “suction” to deliver the sample from the well, through the reaction matrix, and down into the absorbent body. Thus, the absorbent body also acts as a reservoir to hold the sample, and various reagents that are used when the assay is performed. Accordingly, when used in assays where relatively large volumes of liquid are used, the absorbent body should have high absorbent capacity so as to prevent or minimize the possibility of back-flow of sample and reagents from the absorbent body back into the reaction membrane. The absorbent pads are selected for fast absorption of the reaction fluids.

The assay includes a detection agent. The term detection agent refers to the composition of agents that is capable of specifically binding to the antigen and able to form a complex that is detectable. In one embodiment, the detection agent comprising an antigen-specific (e.g. coronavirus-specific) DNA capture sequence (DCS) and a detection reagent, in some embodiments, the detection reagent is linked to a second DCS. The term DNA capture sequences (DCS) is defined as single-stranded oligonucleotides, such as ssDNA, that folds into tertiary structures and bind specifically with the antigen. Herein, the DCS are used interchangeably with the term aptamers, and refer to the polynucleotides that exhibit affinity for a given antigen with selectivity and specificity comparable to antibodies.

In one embodiment, the DSC are altered polynucleotide sequences that have been altered to have high affinity and specificity for a target antigen. In one embodiment the target antigen is SARS-CoV-2. In another embodiment, the DCS have specific affinity to bind the SARS-CoV-2 spike protein. In one embodiment, the DCS are specific for coronavirus. In a suitable embodiment, the SARS-Cov-2 DCS is an LNA-modified DCS. Suitable coronavirus specific DCS are selected from SEQ ID NO: 1-13.

The detection agent can comprise (i) an antigen-specific DNA capture sequence (DCS) linked to the reaction matrix and (ii) a second DCS linked detection reagent or detection molecule. A detection reagent or detection molecule is defined as a material that gives rise to a detectable signal in the presence of the target antigen and DCS. In one embodiment the detection molecule is DCS conjugated to a nanoparticle. The term nanoparticle (NP) refers to particulate materials that are on the nanoscale level (e.g., less than 100 nm in diameter). Suitable nanoparticles can comprise, for example, metals, ceramics, lipids, polymers, among others.

In one embodiment the nanoparticle are metal particles, for example, gold (Au), silver (Ag), platinum (Pt), palladium (Pd). In another embodiment, the nanoparticles are carbon nanotubes or graphene. In another embodiment, the nanoparticles are any suitable nanomaterials that allow for the conjugation to a DCS. In a preferred embodiment, the NP is gold.

In one embodiment the nanoparticles are about 5 nm to about 100 nm in size, preferably about 5 nm to about 50 nm. In one embodiment the nanoparticles are about 10 nm to about 25 nm, for example, but not limited to, 15 nm. The nanoparticles can be of varying sizes to accommodate functionalization to be able to be linked to the DCS. The nanoparticles can be conjugated to DCS by well-known bioconjugation chemistries already documented in the art including, without limitation, thiol chemistry, ‘Click’ chemistry by azide-alkyne cycloaddition such as Copper-Catalyzed Azide-Alkyne cycloaddition (CuAAC), amine conjugation with NHS ester, isocyanate, isothiocyanate, or an anhydride; thiol conjugation with a maleimide or disulfide; carboxylic acid conjugation with a carbodiimide coupling; or other suitably conjugation chemistry.

In one embodiment the detection molecule comprises a detectable tag and a second detectable agent. A detectable tag is defined as an identifiable proxy for the presence of a target molecule. In one embodiment, the detectable tag is biotin and the second detectable agent is streptavidin conjugated to the nanoparticle. In one embodiment the nanoparticle is gold. In one embodiment the nanoparticles are about 5 nm to about 50 nm. In one embodiment the nanoparticles are about 15 nm. The nanoparticles can be conjugated to streptavidin by well-known bioconjugation chemistries already documented in the art including, without limitation, thiol chemistry, ‘Click’ chemistry by azide-alkyne cycloaddition such as Copper-Catalyzed Azide-Alkyne cycloaddition (CuAAC), amine conjugation with NHS ester, isocyanate, isothiocyanate, or an anhydride; thiol conjugation with a maleimide or disulfide; carboxylic acid conjugation with a carbodiimide coupling; or other suitably conjugation chemistry. In one embodiment the streptavidin is conjugated to 15 nm gold nanoparticles in a 10 OD colloidal suspension. In some embodiments, the detection molecule comprises a nanoparticle and is covalently linked to the antigen specific DCS.

In some embodiments, the assay comprises two or more cassettes. In one embodiment, one cassette is the test and the second cassette is a control. The different cassettes can be used, for example, for running a positive or negative control, and/or running more than one different samples at the same time, each cassette having a separate reaction matrix and absorbent pad. In some embodiments, the assay comprises a plurality of cassettes in order to process a plurality of samples at the same time. A plurality of cassettes refers to two or more, three or more, 4, 5, 6, 7, 8, 9, 10, 20, 25, 30, 40, 50, 60, 75, 96, 144, etc., and include any number of cassettes in between or above these numbers.

In one embodiment, the assay is performed with a series of steps. In one embodiment there are four steps comprising deposition of the following solutions on the reaction matrix: the biological sample, the biotin-conjugated DCS, the nanoparticles conjugated to streptavidin, and a buffer wash. In another embodiment, the assay is performed in three steps comprising deposition of the following solutions on the reaction matrix: the biological sample, DCS conjugated to nanoparticles, and a buffer wash. In another embodiment, the methods described herein may be conducted as a one-pot or one step reaction.

An assay showing positive results for the target antigen is indicated by the collection of gold nanoparticles on the reaction matrix. In one embodiment, the collection of nanoparticles may produce a color change on the reaction matrix that may be observable by the unaided eye. In one embodiment the assay is performed alongside a negative control. In one embodiment the negative control is an aqueous solution with a concentration no lower than 0.1 mM and no higher than 10 mM magnesium chloride. In another embodiment, the assay is performed alongside a positive control. In one embodiment a single cassette with a single reaction matrix may be used for the negative or positive control and the test. In another embodiment, the test and the control are performed in separate cassettes on separate reaction matrices. In one embodiment, the disclosure provides a method of detecting an antigen in a sample. The method comprises, a) contacting a sample with a reaction matrix within a gravitational flow through assay described herein, b) allowing the sample to flow through the reaction matrix by gravity; c) contacting the reaction matrix with a solution comprising a second detectable agent; and d) detecting the presence of a complex of the antigen-specific DCS, detection tag, second detectable agent and a nucleic acid sequence from the sample, wherein detection of the complex indicates the presence of antigen in the sample.

In some embodiments, the method further comprises after step b) contacting the matrix with a solution comprising detection reagent, the detection reagent capable of binding to complex of the antigen-specific DCS and antigen to form the detectable complex. In some embodiments, the detection molecule comprises a nanoparticle and is covalently linked to the antigen-specific (e.g., coronavirus specific) DCS. Suitable nanoparticles are described above, and in some embodiments, the nanoparticels are at least 5 nm to about 50 nm. In some embodiments, the method further comprises after step b) a wash step. The wash step allows for any non-bound detectable agents or molecules to flow through the reaction matrix and be absorbed in the absorbent pad, and thus undetectable on the reaction matrix. Suitable wash solutions are described herein.

In some embodiments, a washing step is used to rinse unbound detection agents from the reaction matrix. In some embodiments this washing step is performed using an aqueous liquid. In some embodiments the aqueous liquid is an aqueous buffer solution. In some embodiments the aqueous buffer solution may include, without limitation, phosphate buffer saline (PBS), 2-[bis(2-hydroxyethyl)amino]-2-(hydroxymethyl)propane-1,3-diol (Bis-tris), 2-[4-(2-Hydroxyethyl)-1-piperazine]ethanesulfonic acid (HEPES), 3-Cyclohexylamino-1-propanesulfonic acid (CAPS), 1,4-Piperazinebis(ethanesulfonic acid) (PIPES), N-(2-Acetamido)iminodiacetic acid, N-(Carbamoylmethyl)iminodiacetic acid (ADA), N,N-Bis(2-hydroxyethyl)glycine, Diethylolglycine (Bicine), 2-[Tris(hydroxymethyl)methylamino]-1-ethanesulfonic acid (TES), sodium; 2-morpholin-4-ylethanesulfonate (MES), and other common aqueous buffer solutions well known in the art.

In some embodiments, the detection molecule comprises a detectable tag bound to the DCS and a second detectable agent capable of binding and/or detecting the detectable tag when complexed, the method further comprising after step b) contacting the reaction matrix with a solution comprising the second detectable agent, and optionally subsequently contacting the reaction matrix was a wash solution. In some embodiments, the detectable tag is biotin and the second detectable agent is streptavidin linked to nanoparticles.

In further embodiments, the method is capable of detecting SARS-CoV-2. Thus, in some embodiments, the DCS is an aptamer specific to SARS-CoV-2. In some embodiments, the aptamer comprises a nucleic acid sequence selected from SEQ ID NO:1-13.

In further embodiments, the method may further comprise in step (c) visualizing the detectable complex on the matrix in less than a minute from contacting the matrix with the sample. Visualization can be with the naked eye or with the help of a light or other device.

In yet another embodiment, the disclosure provides a method of detecting coronavirus in a sample, the method comprising: a) contacting a sample with a reaction matrix within a gravitational flow through assay, the gravitational flow through assay comprising: (i) a cassette comprising an absorbent pad; (ii) a reaction matrix comprising nitrocellulose layered on top of the absorbent pad within the cassette, the reaction matrix complexed with a coronavirus-specific DNA capture sequence (DCS); and b) allowing the sample to flow through the reaction matrix by gravity; c) detecting the presence of a complex of the coronavirus-specific DCS and a coronavirus nucleic acid sequence from the sample, wherein detection of the complex indicates the presence of coronavirus in the sample. In a preferred embodiment, the DCS are selected from SEQ ID NO:1-13.

In yet another embodiment, a method of detecting coronavirus in a sample is provided. The method comprises a) contacting a sample with a reaction matrix within a gravitational flow through assay, the gravitational flow through assay comprising: (i) a cassette comprising an absorbent pad; (ii) a reaction matrix comprising nitrocellulose layered on top of the absorbent pad within the cassette, the reaction matrix complexed with a coronavirus-specific DNA capture sequence (DCS) linked to a detection tag; and b) allowing the sample to flow through the reaction matrix by gravity; c) contacting the reaction matrix with a solution comprising a second detectable agent; d) washing the reaction matrix with a solution; and e) detecting the presence of a complex of the coronavirus-specific DCS, detection tag, second detectable agent and a nucleic acid sequence from the sample, wherein detection of the complex indicates the presence of coronavirus in the sample. In some embodiments, step (e) comprises visualizing a change in color on the reaction matrix when the complex is detected.

In yet a further embodiment, the disclosure provides a method of making a gravity flow through assay for coronavirus, comprising: a) conjugating a coronavirus-specific DNA capture sequence (DCS) with a detection tag; b) contacting the coronavirus-specific DCS with a reaction matrix comprising nitrocellulose; c) exposing the reaction matrix to UV light for a sufficient time to link the coronavirus-specific DCS with the reaction matrix; d) layering in a cassette (i) an absorbent pad, (ii) the reaction matrix comprising the DCS linked with a detection tag.

The assay comprises a reaction matrix, defined as a substrate that facilitates the interaction of an antigen with the detection agent. In one embodiment, the coronavirus-specific DNA capture sequence is covalently bound to the reaction matrix. In one embodiment, the DNA capture sequence (DCS) is covalently bound to the reaction matrix. In one embodiment, the DCS by a photoreaction. In one embodiment, the photoreaction occurs with ultraviolet wavelengths of light.

In some embodiments, the photoreaction occurs with exposure to UV light with wavelengths in the range of about 250 to about 370 nm. In some examples, the DCS may incorporate a terminal amine group to facilitate linkage to the reaction matrix. In some examples, a change in color may be seen visually by the naked eye for qualitative “Yes or No” data, also may be read by colorimetry or spectrophotometry to obtain more quantitative data.

In a further embodiment, kits comprising the gravity flow through assay described herein or to perform the methods described herein are provided. For example, in one embodiment, a kit for detecting an antigen is provided, the kit comprising (a) a gravitational flow through assay comprising (i) a cassette containing an absorbent pad; (ii) a porous reaction matrix layered on top of the absorbent pad within the cassette, the reaction matrix comprising nitrocellulose; and (iii) a detection agent comprising (i) an antigen-specific DNA capture sequence (DCS) linked to the matrix and; (b) a solution comprising a detectable molecule; (c) instructions for use. In further embodiments, the kit comprises a wash solution. In further embodiments, the DCS are specific to coronavirus. For example, suitable DCS can comprise one or more polynucleotide sequences of SEQ ID NO:1-13.

### Miscellaneous

Unless otherwise specified or indicated by context, the terms “a”, “an”, and “the” mean “one or more.” For example, “a molecule” should be interpreted to mean “one or more molecules.”

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean plus or minus ≤10% of the particular term and “substantially” and “significantly” will mean plus or minus >10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion additional components other than the components recited in the claims. The term “consisting essentially of” should be interpreted to be partially closed and allowing the inclusion only of additional components that do not fundamentally alter the nature of the claimed subject matter.

All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

All references, including publications, patent applications, and patents, cited herein are hereby incorporated by reference to the same extent as if each reference were individually and specifically indicated to be incorporated by reference and were set forth in its entirety herein.

Preferred aspects of this invention are described herein, including the best mode known to the inventors for carrying out the invention. Variations of those preferred aspects may become apparent to those of ordinary skill in the art upon reading the foregoing description. The inventors expect a person having ordinary skill in the art to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### EXAMPLES

### Example 1. Modification of N-antigen DNA Captures to Detect SARS-CoV-2

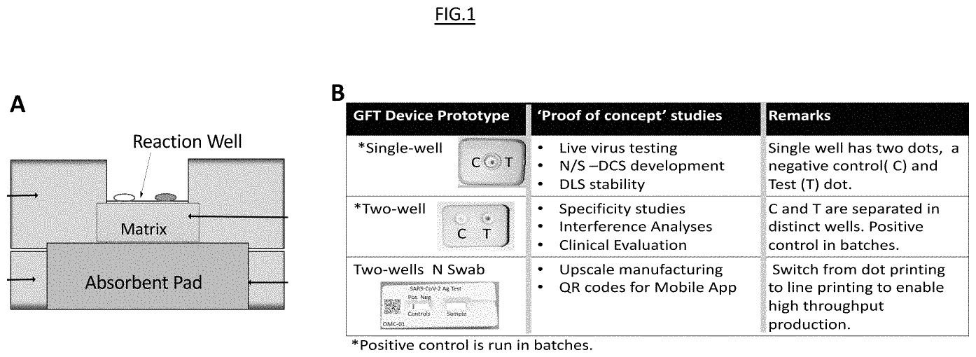

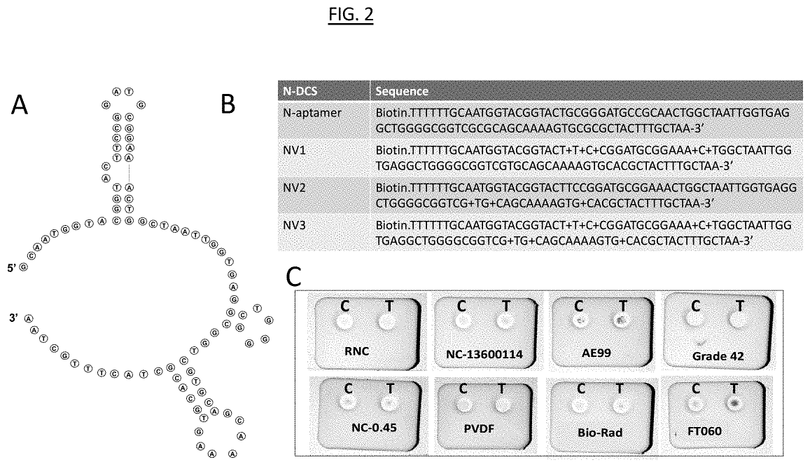

To develop the best DNA Capture Sequence (DCS) for the SARS-CoV-2 N antigen, a published N aptamer sequence reported by Chen et al. (Virologica sinica, 2020:35:351- 354) was selected as the basal DNA capture and a positive control for N antigen was used (N-DCS). To increase stability and storage half-life of the DCN, the locked nucleic acids (LNAs) were incorporated into the N-DCS. These modified bases contain a methylene bridge bond (orange) between the 2′ oxygen and the 4′ carbon of the pentose ring. LNAs are known to increase hybrid melting temperature (Tm), and improve docking specificity, sequence stability, nuclease resistance, and mismatch discrimination. The FIG. 2A-B shows the sequences of the DNA captures that were synthesized. The N-DCSs were tested for binding to SARS-COV-2 N protein or RNA using a 3D-printed GFT device with built-in wells for both control (C) and test sample (T).

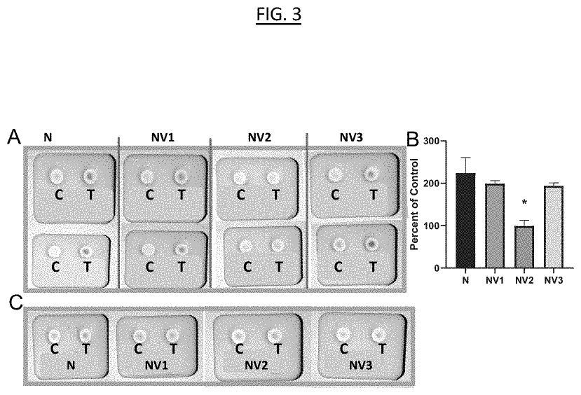

A GFT system rapid test was established using nitrocellulose membranes including FT060 (Matrix 1) and BioRad (Matrix 2) as reaction matrix layered on top of absorbent cellulose paper sandwiched inside a plastic cassette to ensure uniform contact (FIG. 3). To determine the optimal membrane matrix for the test a number of membranes of different types (cellulose, nitrocellulose, nylon, PVDF, etc.) were compared. To test each membrane 5 μL of sample was placed onto the membrane containing 5×LOD inactivated virus. Next, 5 μL 100 μM Biotin-NV3 Aptamer was added onto the test well and 5 uL aptamer folding buffer was added onto the control well followed by 5 μL Streptavidin-gold (size 15 nm, 10 OD from Cytodiagnostics) in both wells. The cassette was washed with 3 drops of wash buffer and the color development was observed. The results showed that only the FT060 nitrocellulose membrane generated maximal binding reaction followed by AE99. To test whether the DNA capture sequences differed in their ability to detect N antigens, Sequence variants of the N aptamers were tested on the select matrix-1 i.e., FT060 as previously described. Signal quantitation revealed that both the NV1 and NV3 performed approximately equal to the original N sequence while NV2 was not able to detect the virus (FIG. 3).

### Example 2. Molecular Docking Analysis to Identify DNA Captures for SARS-CoV-2 S Antigen

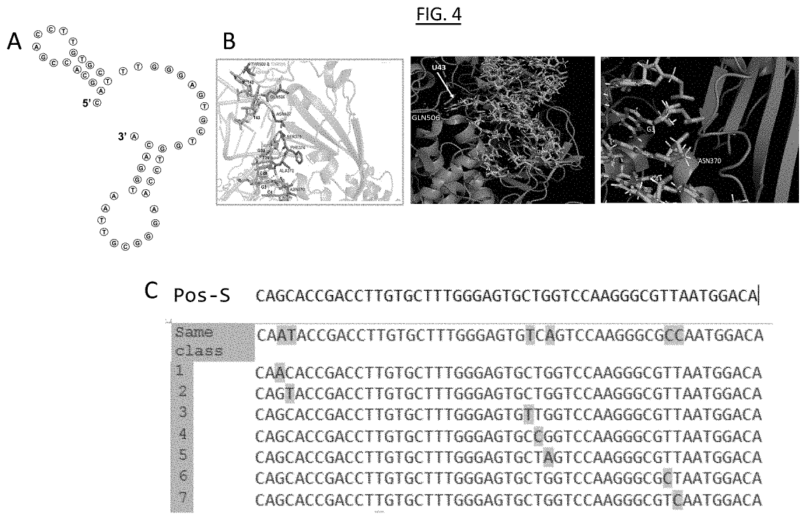

Since N antigen-based test may not distinguish between SARS-CoV-1 and the SARS-CoV-2 N, to develop a test more specific for SARS-CoV-2, Spike (S) protein was used in addition to N protein. To identify the best DNA Capture Sequence (DCS1) for the SARS-CoV-2 S antigen, a published we S aptamer sequence reported by Song et. al. (Analytical Chemistry, 2020: 92, 9895-9900) was selected as the basal DNA capture and a positive control (Pos-S). Simulated interaction modeling and competition experiments using molecular docking suggested that this aptamer may have partially identical binding site at ACE2 on SARS-CoV-2 RBD. Since better binding is required for in vitro diagnostic tests, DCS1 was further modified by substituting nucleotides for the corresponding nucleotide in the same class (purine for purine or pyrimidine for pyrimidine). Sequences “same class” and 1-7 contain substitutions in the areas of the aptamer predicted to interact with the S protein while sequences A-C contain substitutions in the areas between the predicted interacting regions (FIG. 4). The secondary structures of these sequences were predicted using centroid fold and PDB files of the predicted 3D structure of each sequence were generated using RNA composer. Energy minimization of the 3D structure was performed in the molecular operating environment (MOE) software and the top 3 poses of each sequence were selected for docking to the RBD of S protein (6VSB). Docking results were visualized using PyMOL. DCSs were purchased from Integrated DNA technologies (IDT) with biotin conjugated to either the 5-prime or 3-prime end, whichever was predicted by molecular docking to be located furthest from the S protein within the S-aptamer complex, to minimize potential interference caused by the biotin molecule.

### Example 3. Testing of the S antigen-DNA Captures for Detection of SARS-CoV-2 at Lower Concentration of the Virus

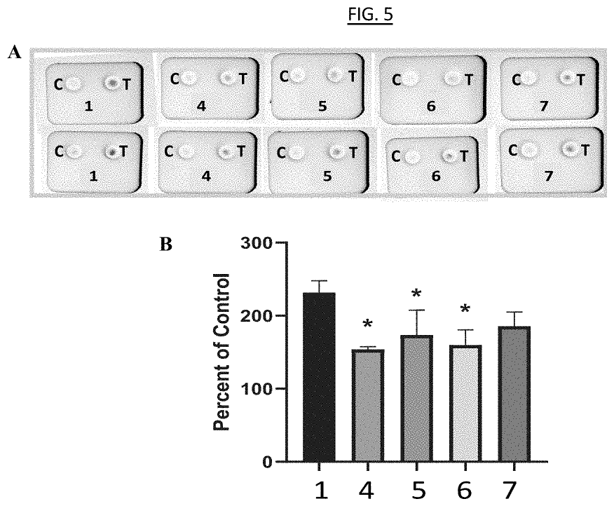

To test whether the S antigen-DNA captures can be used for detection of SARS-CoV-2 at lower concentration of the virus, four candidate sequences were tested using FT060 membrane and 5×LOD inactivated SARS CoV-2, as previously described. Sequences 1, 4, 5, and 6 all generated significantly lower signal than the parental sequence as quantitated by imageJ (n=2) p≤0.05 (FIG. 5). Sequence #7 showed a slightly lower signal than the parental sequence although this difference was not significant.

### Example 4. Evaluation of S Antigen-DNA Captures with Linkage to Matrix Detection of SARS-CoV-2: Comparison of Matrixes

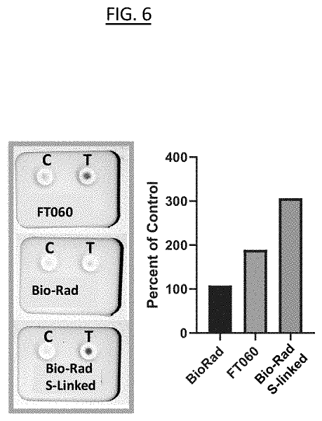

Because the detection depends on the membrane surface retention of the gold conjugates in presence of the virus, the specific hydrophilicity/hydrophobicity and flow rate of the membrane which is determined by both its composition and backing are crucial to the function of the test. The protein binding property of nitrocellulose matrix likely aids in this surface retention. Hence, to improve the matrix, an aptamer-based DNA capture sequence was developed and linked to the membrane to improve virus detection. To test this idea, the DNA capture sequence containing an amine linker was synthesized and was linked to membrane by adding 5 uL of 2 ug/mL amine conjugated S DNA capture sequence and exposed to the matrixes (Matrix-1 being FT060 and Matrix 2=Biorad) to 25 mJ/cm\hat{ }2 of 254 nm UV in a Stratalinker. The test was conducted as described for NV3 (FIG. 6). Signal generated by the 5×LOD SARS COV-2 in the test was quantitated using ImageJ. The images of each test were first converted to a hue/saturation/brightness (HSB) stack and the stack was split into its individual components. The saturation layer was selected, and the control and test wells were defined as regions of interest using the circular selection tool. The integrated density was measured within both the control and test regions and the value obtained from the test well was converted to a percent of the control value.The Matrix 2 without S-linking does not show any meaningful signal in the test well compared to the background in control. The FT060 membrane shows an approximately 2× greater signal intensity in test compared to control while the Bio-Rad with S aptamer linked to the membrane shows approximately a 3Δ increase.

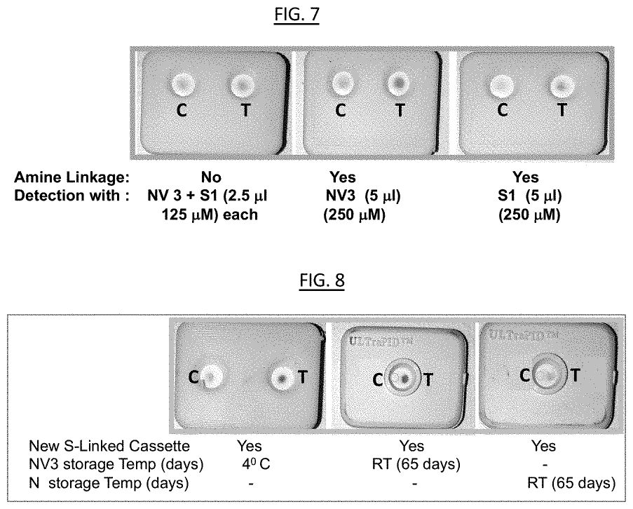

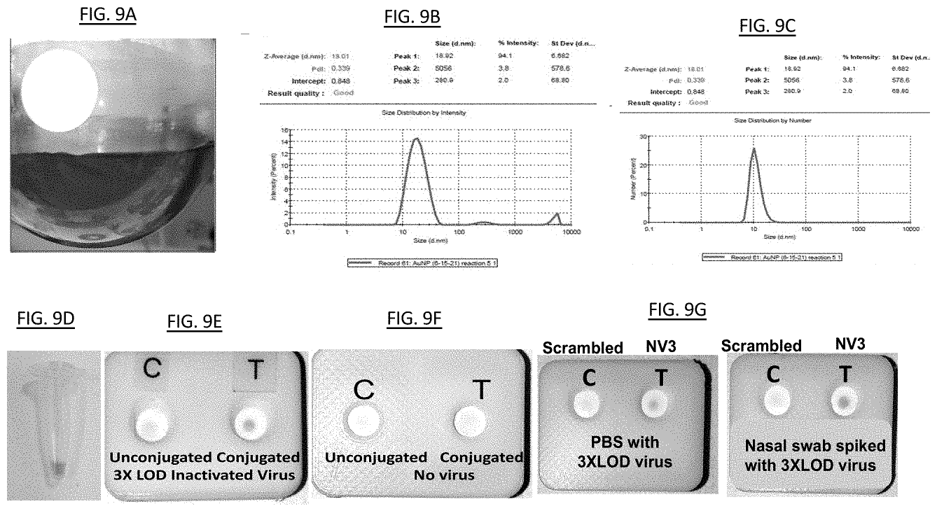

To further confirm the utilization of Matrix 2 (BioRad), it was tested using a mixture of NV3 and S aptamers to detect inactivated virus at 5×LOD. The light signal observed in the test well indicates that this is not a suitable detection method. Of all detection methods tested, Bio-Rad membrane was only observed to detect the virus after amine conjugated S aptamer has been linked to it via the UV method. The S-Linked Bio-Rad membrane was also tested using biotinylated S aptamer for detection (FIG. 7). The signal generated was lighter than the signal generated when NV3 is used for detection. This is likely due to the capture and detection antibodies both competing for the same binding epitope in the sample. Together these results show that the S-linked Matrix-2 (Bio-Rad) using the NV3 aptamer for detection produced the strongest signal in the presence of inactivated SARS CoV-2 virus. The NV3 also has the advantage of increased stability and shelf life provided by its substitutions with locked nucleic acids.

### Example 5: Determining Stability of the NV3 DNA Capture Sequences

To determine whether the substituting and adding locked nucleic acids in the DCSs increases stability, S linked cassette and N aptamer stability were tested by storing both cassettes and aptamers at room temperature. Both freshly prepared S linked cassettes and S linked cassettes stored at room temperature for 65 days retained similar ability to detect SARS CoV-2 at 3×LOD. Likewise, signal generated by NV3 stored at room temperature for 65 days remained strong. However, the original N aptamer sequence showed greatly reduced signal after 65 days at room temperature as compared to NV3. This indicates possible un-folding or degradation of the N aptamer while the NV3 remained stable (FIG. 8).

### Example 6. Testing of the N antigen-DCS for Detection of SARS-CoV-2 N by Linking DCS with Gold Nanoparticles

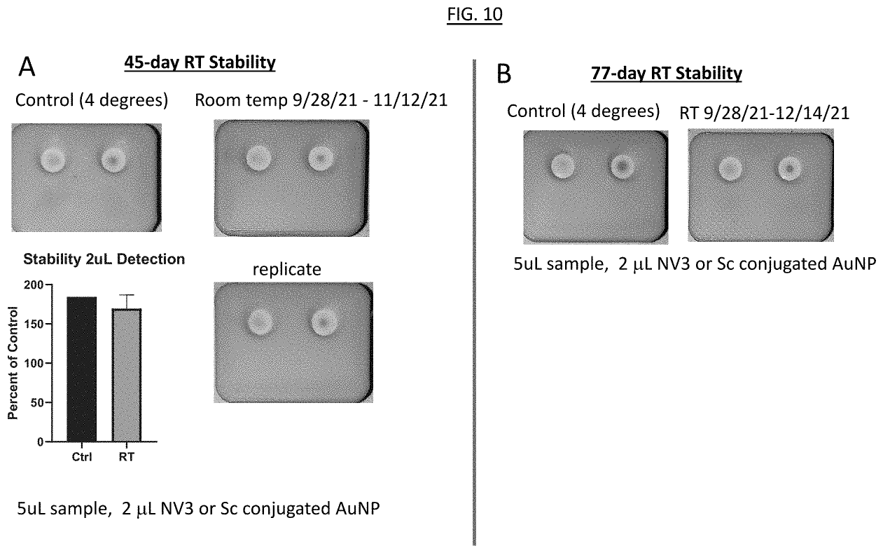

Because of the high background inherent with biotin-streptavidin amplification system and potential to increase false positives and toward decreasing the number of steps involved in the test, the N antigen DCS was directly conjugated to the gold nanoparticles synthesized by the Turkevich method using a lipoic acid linker. NV3 aptamer conjugate was prepared for detection of virus and scrambled NV3 was prepared as a control for non-specific binding. Briefly, the N aptamer sequence with 5′ amine group was modified by substituting T #60, C #62, and C #73 with IDT's Affinity Plus locked nucleotides. The folded DCSs were conjugated to gold nanoparticles using a lipoic acid linker. Lipoic acid was reacted with EDC in a 1:1 molar ratio for 2 hr to activate the carboxylic acid group. Then the activated lipoic acid was added to the folded aptamer in a 1:1 molar ratio and allowed to react for 1 hr while shaking. The lipoic-aptamer complex was conjugated to gold nanoparticles through the affinity of the thiol group for the gold surface. The complex was added to 1 mL gold nanoparticles and stirred overnight. 2M NaCl was added to bring the NaCl concentration to 3.6 mM and the reaction was stirred for 1 h. This process was repeated to bring the NaCl concentration to 7.2 mM, then 10.8 mM. Finally, the particles were collected by centrifuging at 7,000 g for 10 min and resuspended in PBS containing 1% BSA and 20% glycerol. To examine the functioning of the DCS conjugated with gold nanoparticles, the tests were performed on S-linked matrix with unconjugated and conjugated DCSs with 4 uL of gold nanoparticles. The results shown in FIG. 10A-D provide evidence for a successful synthesis of gold NPs. The size of the gold nanoparticles obtained was measured by DLS directly from reaction without sonication, filtration, or centrifugation. Most of the particle size distribution was centered around the peak of approximately 19 nm with a PDI of 0.34. The 19 nm peak encompassed ˜94% of the signal and diluted to the appropriate concentration. Also, the results showed directly conjugating the DCS to gold nanoparticles can specifically detect the N antigen in inactivated virus. There was no background color in absence of the virus (control).

Further to test specificity of the reaction, Scrambled control conjugated gold nanoparticles were compared to NV3 conjugated gold nanoparticles on the S-Linked Matrix-2 (Bio-Rad membrane). Very low background was observed from the scrambled control in either PBS sample buffer or nasal swab spiked with inactivated virus suggesting high specificity of the test. In addition the stability of gold NPs were tested at day 45 and day 77 after its storage at room temperature (FIG. 10) and the results show that the NPs were stable at room temperature.

### Example 7. Analyses of Limit of Detection for the Test

The Limit of Detection (LoD) studies determine the lowest detectable concentration of SARS-CoV-2 at which approximately 95% of all (true positive) replicates test positive. The LoD was determined by limiting dilution studies using characterized [Heat-inactivated SARS-CoV-2 (BEI: NR-52286)].

LoD was determined using limiting dilutions of heat-inactivated SARS-CoV-2 (BEI: NR-52286) (Table 1). NR-52286 is a preparation of SARS-Related Coronavirus 2 (SARS-CoV-2), isolate USA-WA1/2020 that has been inactivated by heating to 65° C. for 30 minutes. The material was supplied frozen at a concentration of 1.6×10⁵ TCID50/mL. In this study virus stock was diluted 32 times to 5×10³ TCID50/mL and the swab was spiked with approximately 50-μL of the virus dilution in PBS. The LoD was determined using following steps: A 10-fold dilution of the heat inactivated virus was made in PBS and processed for each study as described above. These dilutions were tested in triplicate. The lowest concentration demonstrating 20 of 20 positives was chosen for LoD range finding. Based on this testing using Matrix-1 (FT060) matix, the concentration chosen was TCID50 of 5×10² PFU per mL. Serial dilutions of the characterized SARS-CoV-2 were then tested in [3] replicates. The lowest concentration at which all [20] replicates were positive was treated as the tentative LoD for each test. The LoD of each test was then confirmed by testing [The concentration 3.21×10² dilution was tested twenty (20) times. Twenty (20) of twenty (20) results were positive. Based on this testing the concentration was confirmed as TCID50 of 5.38×10² PFU per mL] with concentrations at the tentative limit of detection. The final LoD of each test was determined to be the lowest concentration resulting in positive detection of [20 of 20 replicates].

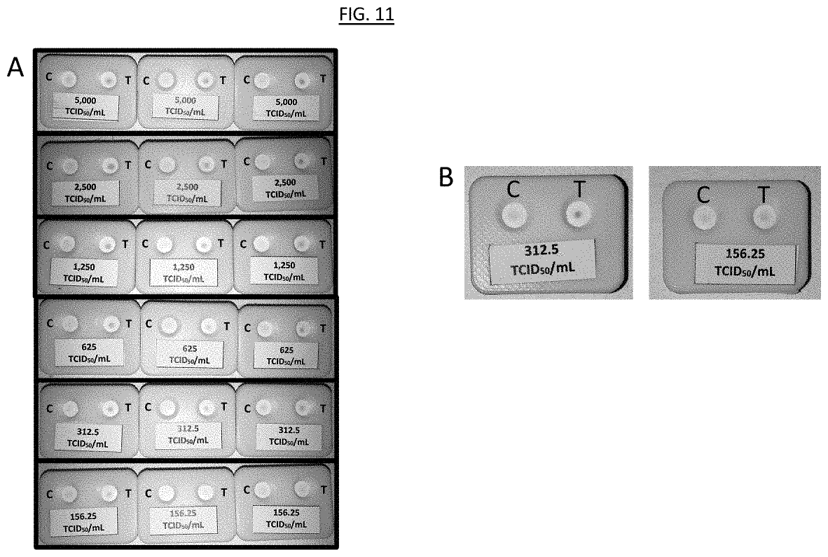

In addition to Matrix-1, the limit of detection (LOD) was determined using S-Linked Matrix-2 (Bio-Rad membrane) and NV3 (test) or scrambled (control) aptamer conjugated gold nanoparticles. A serial dilution of inactivated SARS CoV-2 virus was used as sample in both the test and control wells (5 uL) and tests were performed in triplicate. The lowest concentration of virus that was reliably detected by the test was a concentration of 312.5 TCID50/mL (FIG. 11A). This is an improvement over the 538 TCID50/mL achieved with the FT060 membrane (FIG. 11 B).

TABLE 1
Determination LoD of the Test using Matrix-1 and biotin-
streptavidin amplification system on prototype 2 device.
LoD Range Detection

| Concentration (TCID50/mL) | Positive test | |
|---|---|---|
| 17,200 | 3/3 | Serial |
| 8600 | 3/3 | Dilution |
| 4300 | 3/3 | ↓ |
| 2150 | 3/3 | |
| 1075 | 3/3 | |
| 537.5 | 3/3 | |
| 268.75 | 0/3 | |

Confirmation of LoD

| Concentration (TCID50/mL) | Positive test |
|---|---|
| 538 | 20/20 |
| 430 | 1/3 |

### Example 8. Characterization of Viral Cross-Reactivity (Analytical Specificity) of the Test

To determine analytical specificity of this test, cross-reactivity studies was performed to demonstrate that the test does not react with related pathogens, high prevalence disease agents, and normal or pathogenic flora that are reasonably likely to be encountered in the clinical specimen. For this purpose, the organisms shown in the table below were wet tested using the Matrix-1 (FT060) and four step protocol involving sample added to amine S-linked to matrix followed by biotinylated NV3 DCS, and then streptavidin-conjugated to gold and a wash. For wet testing, concentrations of 10⁵ pfu/ml or higher for viruses was used. The results of our bacterial interference studies are shown in Table 2.

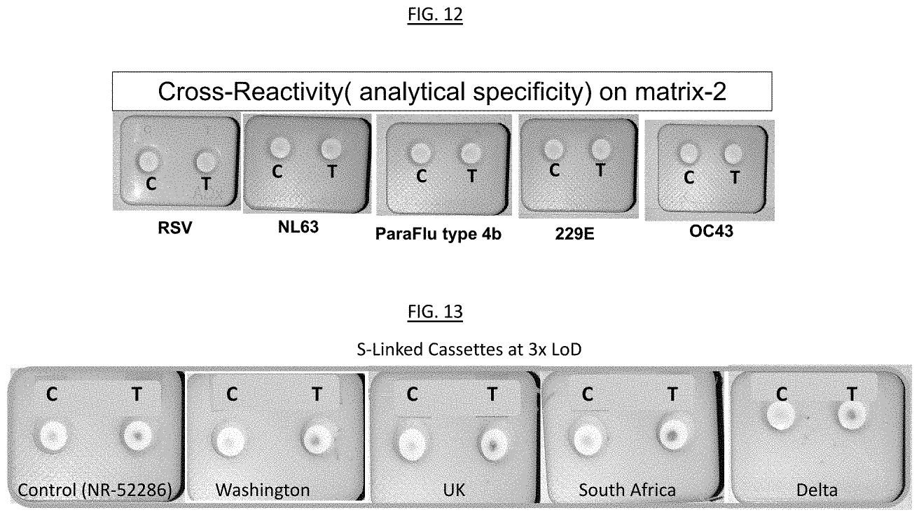

Further, a cross-reactivity study was also performed using 1×10⁵ TCID50/mL samples of common respiratory viruses using Matrix 2 and a 3-step protocol that does not use biotin-streptavidin amplification. No cross-reactivity was detected with the viruses tested (FIG. 12). Also, the potential of the OMC-01 test to detect different mutant isolates was examined five different available mutant isolates including the delta isolate. Thus, study was performed using 5 μL samples of 1×10⁵ TCID50/mL common respiratory viruses on amine S-linked Matrix-2 and the 3-step gold conjugated NV3 detection method. All different isolates were equally detected by the test (FIG. 13).

TABLE 2
Characterization of viral cross-reactivity (analytical specificity) of the test

| Virus | Strain | Codes | Concentration | Cross-Reactivity | Interference |
|---|---|---|---|---|---|
| Coronavirus | 229e | V001 | 1 × 10⁵ TCID50/ML | No Cross-Reactivity | No Interference |
| Coronavirus | OC43 | V002 | 2 × 10⁴ TCID50/ML | No Cross-Reactivity | No Interference |
| Coronavirus | NL63 | V003 | 8 × 10³ TCID50/ML | No Cross-Reactivity | No Interference |
| Influenza A H3N2 | Brisbane/10/2007 | V004 | 1 × 10⁵ TCID50/ML | No Cross-Reactivity | No Interference |
| Influenza A H1N1 | Brisbane/59/2007 | V005 | 1 × 10⁵ TCID50/ML | No Cross-Reactivity | No Interference |
| Influenza B | Brisbane/60/2008 | V006 | 1 × 10⁵ TCID50/ML | No Cross-Reactivity | No Interference |
| Parainfluenza | Type 2 | V007 | 1 × 10⁵ TCID50/ML | No Cross-Reactivity | No Interference |
| Parainfluenza | Type 3 | V008 | 1 × 10⁵ TCID50/ML | No Cross-Reactivity | No Interference |
| Parainfluenza | Type 4a | V009 | 8 × 10³ TCID50/ML | No Cross-Reactivity | No Interference |
| Parainfluenza | Type 4b | V010 | 1 × 10⁵ TCID50/ML | No Cross-Reactivity | No Interference |
| Enterovirus | Type 68 US/MO/14-18949 | V011 | 1 × 10⁵ TCID50/ML | No Cross-Reactivity | No Interference |
| Human Rhinovirus | Type 60 2268-CV37 | V012 | 1 × 10⁵ TCID50/ML | No Cross-Reactivity | No Interference |
| Human Metapneumovirus | TN/83-1211 | V013 | 1 × 10⁵ TCID50/ML | No Cross-Reactivity | No Interference |
| Respiratory Syncytial | A2 | V014 | 1 × 10⁷ TCID50/ML | No Cross-Reactivity | No Interference |
| Virus | | | | | |

### Example 9. Characterization of Bacterial Cross-Reactivity (Analytical Specificity) of the Test

To determine analytical specificity of this test, cross-reactivity studies was performed to demonstrate that the test does not react with related bacterial pathogens, high prevalence disease agents, and normal or pathogenic flora that are reasonably likely to be encountered in the clinical specimen. For this purpose, the organisms shown in the table below were wet tested using the Matrix-1 (FT060) and four step protocol involving four step protocol involving sample added to amine-S-linked to matrix followed by biotinylated NV3 DCS, and then streptavidin-conjugated to gold and a wash. For wet testing of microbial interference, samples spiked at a low (3×LoD) SARS-CoV-2 concentration and a high interferent level (preferably microorganisms), to represent the worst-case scenario, with a minimum of 3 replicates. The interferent microorganisms were tested individually. The results of our bacterial interference studies are shown in Table 3.

TABLE 3
Specificity of Test: Bacterial Cross-Reactivity/Interference by Wet Testing

| Bacteria/parasites | Strain | Codes | Concentration | Cross-Reactivity | Interference |
|---|---|---|---|---|---|
| Candida albicans | ATCC | V018 | 1 × 10⁶ CFU/mL | No Cross-Reactivity | No Interference |
| | MYA-2876 | | | | |
| Haemophilus infuenzae | ATCC 19418 | V020 | 1 × 10⁶ CFU/mL | No Cross-Reactivity | No Interference |
| Streptococcus pyogenes | ATCC 19615 | V022 | 1 × 10⁶ CFU/mL | No Cross-Reactivity | No Interference |
| Legionella pneumophila | ATCC 33152 | V023 | 1 × 10⁶ CFU/mL | No Cross-Reactivity | No Interference |
| Streptococcus pneumoniae | ATCC 49619 | V024 | 1 × 10⁶ CFU/mL | No Cross-Reactivity | No Interference |
| Staphylococcus epidermidis | ATCC 14990 | V026 | 1 × 10⁶ CFU/mL | No Cross-Reactivity | No Interference |
| Staphylococcus aureus | ATCC 29213 | V027 | 1 × 10⁶ CFU/mL | No Cross-Reactivity | No Interference |

### Example 10. Characterization of Test Interference by Endogenous Substances

To determine test interference by any endogenous host factors or treatments that may be commonly administered to alleviate symptoms associated with SARS CoV-2 infection, studies was performed to demonstrate that the test does not react with any of these factors that are reasonably likely to be encountered in the clinical specimen. For this purpose, the organisms shown in the table below were wet tested using the Matrix-1 (FT060) and four-step protocol involving four step protocol involving sample added to amine-S-linked to matrix followed by biotinylated NV3 DCS, and then streptavidin-conjugated to gold and a wash. For wet testing of interference by endogenous substances, samples spiked at a low (3×LoD) SARS-CoV-2 concentration and a high interferent level (preferably microorganisms), to represent the worst-case scenario, with a minimum of 3 replicates. The interferent microorganisms were tested individually. The results of our endogenous substance interference studies are shown in Table 4.

TABLE 4
Summary of Drug Cross-reactivity/Interference studies by Wet Testing

| Substances | Active Ingredient | Concentration | | Codes | Cross-Reactivity | Interference |
|---|---|---|---|---|---|---|
| Blood (human) | Blood | 4% | v/v | D001 | No | No |
| Purified mucin | Mucin protein | 0.5% | | D002 | No | No |
| protein | | | | | | |
| Vicks ® Vapocool | Benzocaine, Menthol | 1.5 | mg/mL | D003 | No | No |
| Ayr Nasal Gel | Saline | 5% | v/v | D004 | Yes | Yes |
| CVS Nose Drops | Phenylephrine | 15% | v/v | D005 | No | No |
| | hydrochloride | | | | | |
| Afrin ® - nasal spray | Oxymetazoline HCI | 15% | v/v | D006 | No | No |
| NasalCrom ® Spray | Cromolyn Sodium | 15% | v/v | D007 | No | No |
| Zicam ® Cold | Galphimia glauca, | 5% | v/v | D008 | No | Yes |
| Remedy | Luffa operculate, | | | | | |
| | Sabadilla | | | | | |
| Homeopathy Alkalol | | 1:10 dilution | | D009 | No | No |
| Chloraseptic ® max | Phenol | 15% | v/v | D010 | No | No |
| Tobramycin | Tobramycin | 4 | ug/mL | D011 | No | No |
| Mupirocin | Mupirocin | 10 | mg/mL | D012 | No | No |
| CVS Nasal Spray | Fluticasone | 5% | v/v | D013 | No | No |
| Oseltamivir | Oseltamivir | 5 | mg/mL | D014 | No | No |

### Example 11. Evaluation of Clinical Sensitivity of Test

To conduct the clinical evaluation of the test, a clinical study (IRB #1708773) was conducted at the Sarasota memorial Hospital Emergency Clinic. Ninety-four patients were recruited from those wanted a SARS-CoV-2 test and were able to sign an informed consent. A team of 4 operators (3 nurses and 1 laboratory personnel) were used to run the assays. Patients who have been diagnosed with COVID-19 or have the suspicion of being infected with COVID-19 virus were enrolled. Inclusion criteria are age of 18 or greater and who are able and willing to consent. Two swab samples were collected by nursing staff: a nasopharyngeal swab for PCR analysis as per standard of care guidelines, and a second nasal swab by inserting swab into left and right nostrils of subjects at the time of patient admission. For the latter the testing was performed within 15 mins. The PCR assay was performed in the hospital stat-Lab that uses HOLOGIC Aptima SARS CoV-2, Biofire Respiratory 2.1 Panel and Genexpert SARS CoV-2/FLU/RSV test kits.

Thus, prospectively a total number 94 samples tested for both PCR and the ultra-rapid test, designated OMC-01 described here using 4 step procedures. The Nasal Swab samples were directly used for OMC-01 testing. Analysis of these clinical samples at PoC thus far have shown that the test has an overall >97.9% sensitivity and specificity (Table 5).

In addition, OMC-01 test was directly compared head-to-head with a commercial rapid test, i.e., Quidel test, which also used a nasal swab for the test in an urgent care setting (N=39). The analysis of results is shown in Table 6. All rests matched between the two tests except one subject, who was with COVID symptoms for which the OMC-01 test showed a positive test result, but the Quidel test was negative. However, the PCR result was not available for this subject.

TABLE 5
The summary statistic of sensitivity of ultra-rapid
One-Minute CoviTest in clinical samples

| | RT-PCR Assay | | |
|---|---|---|---|
| Rapid Test | Positive | Negative | Total |
| OMC >= 0.935 | 29 | 63 | 92 |
| Assay < 0.935 | 2 | 0 | 2 |
| Total | 31 | 63 | 94 |
| Summary statistics | Percent | *Lo Limit | Hi Limit |
| Positive Agreement PPA | 93.50% | 79.30% | 98.20% |
| Negative Agreement | 100.00% | 94.30% | 100.00% |
| PNA | | | |
| Overall Agreement POA | 97.90% | 92.60% | 99.40% |

*95% Confidence Interval

| | | | PCR | % |
|---|---|---|---|---|
| Test | Signal Intensity | | Match | Agreement |
| Strong | >50% | Increase | 4/4 | 100 |
| Medium | 20-50% | Increase | 12/12 | 100 |
| Weak | <20% | increase | 86/88 | 97.7 |

LoD: 540 TCID50/ml (~54 ng/ml
Specificity of test: no viral, bacterial, drug Interference

TABLE 6
Head-to-head comparison of sensitivity of ultra-rapid One-
Minute CoviTest with the Quidel test in clinical samples

Quidel test

| OMC-01 test | Positive | Negative | Total |
|---|---|---|---|
| Positive | 8 | 1 | 9 |
| Negative | 0 | 29 | 29 |
| | 8 | 30 | 38 |

| Summary statistics | Percent | Lo Limit | Hi Limit |
|---|---|---|---|
| Positive Agreement | 100.00% | 67.60% | 100.00% |
| PPA | | | |
| Negative Agreement | 96.70% | 83.30% | 99.40% |
| PNA | | | |
| Overall Agreement POA | 97.40% | 86.50% | 99.50% |

Alternative combinations and variations of the examples provided will become apparent based on this disclosure. It is not possible to provide specific examples for all of the many possible combinations and variations of the embodiments described, but such combinations and variations may be claims that eventually issue.

### Example 12. Detection of Antigens in Finger-Prick Blood

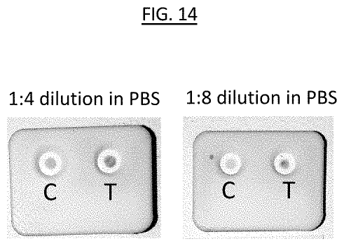

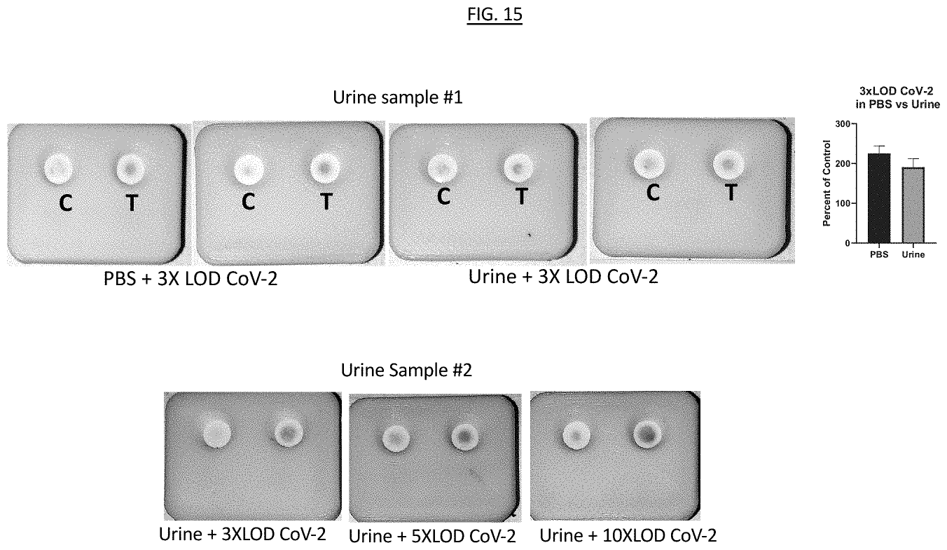

To examine whether a desired antigen can be directly detected in blood we examined spiking the antigen in the finger prick blood with or without the dilution with saline. Briefly, blood was collected by finger prick and added 5×, or 10×LOD (600, or 3,200 TCID50/mL) UV inactivated SARS CoV-2 delta strain was added to the diluted blood. 5 uL of blood spiked with virus was dispensed onto each well of a Bio-Rad S aptamer linked cassette. 2 uL detection reagent (NV3) was added to the test well and 2 uL scrambled control reagent was added to the control well followed by 2 drops wash buffer to each well. The results are shown in FIG. 14.

The results showed that when undiluted blood was passed through the matrix, blood did not flow through the matrix. Also, upon adding 1 to 2 dilution the background was high. However, with 1:4 dilution, the test provided a result that is positive, but background was reduced vs 1:2 dilution. A dilution of spiked blood at 1:8 resulted in the best signal to noise ratio and it brings final concentration of virus to 2.5×LOD and result is more clearly positive due to lower background. Thus, these results indicate that antigen can be detected in the blood.

### Example 13. Detection of Antigens in Urine Samples

To test whether our platform can be used to detect biomarker antigens present in the urine, we compare our standard approach of detecting antigen using PBS buffer urine samples spiked with known concentration of inactivated virus antigen. The results showed that the platform was able detect this inactivated viral antigen with 3×LOD, which is like that of the viral detection from the nasal swab (FIG. 15).

### References: 54

1. Hui, D. S., et al., The continuing 2019-nCoV epidemic threat of novel coronaviruses to global health—The latest 2019 novel coronavirus outbreak in Wuhan, China. International journal of infectious diseases: IJID: official publication of the International Society for Infectious Diseases, 2020. 91: p. 264-266.

2. Coronavirus Disease 2019 (COVID-19)—Mayo Clinic. Available from: https://www.mayoclinic.org/diseasesconditions/coronavirus/symptoms-causes/syc-20479963.

3. COVID 19 aptamers—BasePair Biotechnologies Available from: https://www.basepairbio.com/covid19/.

4. Coronavirus and COVID-19: What You Should Know—WebMD.

5. Döhla, M., et al., Rapid point-of-care testing for SARS-CoV-2 in a community screening setting shows low sensitivity. Public Health, 2020. 182: p. 170-172.

6. How the SARS-CoV-2 EUA Antigen Tests Work—American Society for Microbiology 2020; Available from: https://asm.org/Articles/2020/August/How-the-SARS-CoV-2-EUA-Antigen-Tests-Work.

7. Scohy, A., et al., Low performance of rapid antigen detection test as frontline testing for COVID-19 diagnosis. Journal of clinical virology: the official publication of the Pan American Society for Clinical Virology, 2020. 129: p. 104455-104455.

8. Zhang, Y., B. S. Lai, and M. Juhas, Recent Advances in Aptamer Discovery and Applications. Molecules (Basel, Switzerland), 2019. 24(5): p. 941.

9. Gopinath, S.C.B., et al., Aptamer-based ‘point-of-care testing’. Biotechnology Advances, 2016. 34(3): p. 198-208.

10. Ross, G. M. S., G. I. Salentijn, and M. W. F. Nielen, A Critical Comparison between Flow-through and Lateral Flow Immunoassay Formats for Visual and Smartphone-Based Multiplex Allergen Detection. Biosensors, 2019. 9(4): p. 143.

11. Chen, P., et al., Paper-based Vertical Flow Immunoassay (VFI) for detection of bio-threat pathogens. Talanta, 2019. 191: p. 81-88.

12. Jiang, N., et al., Lateral and Vertical Flow Assays for Point-of-Care Diagnostics. 2019. 8(14): p. 1900244.

What is claimed is:

1. A direct gravitational flow through assay for detecting an antigen in a sample comprising:
(a) a cassette comprising an absorbent pad;
(b) a reaction matrix layered on top of the absorbent pad within the cassette, the reaction matrix comprising nitrocellulose backed with cellulose or polymer membrane, and
(c) a detection agent comprising (i) an antigen-specific DNA capture sequence (DCS) linked to the matrix and (ii) a second DCS linked detection reagent;
wherein when the assay is contacted with a biological sample capable of gravitational flow through the matrix and absorbent pad, and wherein the formation of a complex between the detection reagent and sample, DCS and an antigen encoding sequence on the matrix within the cassette detects the presence of the antigen within the assay.

2. The gravitational flow through assay of claim 1, wherein the matrix comprises cellulose or polyester supported nitrocellulose membrane with a pore size from about 0.4 μm to about 0.8 μm.

3. The gravitational flow through assay of claim 1, wherein the nanoparticles are at least 5 nm to 40 nm in diameter.

4. The gravitational flow through assay of claim 1, wherein the detection molecule comprises a detectable tag bound to the DCS and a second detectable agent capable of binding and/or detecting the detectable tag when complexed.

5. The gravitational flow through assay of claim 4, wherein the detectable tag is biotin, and the second detectable agent is streptavidin, and wherein the streptavidin is conjugated to a nanoparticle.

6. The gravitational flow through assay of claim 1, wherein the detection molecule comprises a nanoparticle and is covalently linked to the antigen specific DCS.

7. The gravitational flow through assay of claim 1, wherein the assay comprises at least two cassettes, wherein one cassette is a control.

8. The gravitational flow through assay of claim 1, wherein the antigen-specific DNA capture sequence is specific to SARS-CoV-2.

9. The gravitational flow through assay of claim 8, wherein the SARS-Cov-2 DCS is an LNA-modified DCS.

10. The gravitational flow through assay of claim 8, wherein the SARS-Cov-2 DCS is to the nucleocapsid (N) protein or spike (S) protein.

11. The gravitational flow through assay of claim 8, wherein the DCS comprises a sequence selected from SEQ ID NO: 1-13.

12. The gravitational flow through assay of claim 1, wherein the assay comprises a plurality of cassettes in order to process a plurality of samples at the same time.

13. A method of detecting an antigen in a sample, the method comprising:
a) contacting a sample with a reaction matrix within a gravitational flow through assay of claim 1,
b) allowing the sample to flow through the reaction matrix by gravity;
c) contacting the reaction matrix with a solution comprising a second detectable agent; and
d) detecting the presence of a complex of the antigen-specific DCS, detection tag, second detectable agent and a nucleic acid sequence from the sample, wherein detection of the complex indicates the presence of antigen in the sample.

14. The method of claim 13, wherein method further comprises after step b) contacting the matrix with a solution comprising detection reagent, the detection reagent capable of binding to complex of the antigen-specific DCS and antigen to form the detectable complex.

15. The method of claim 13, wherein the detection molecule comprises a nanoparticle and is covalently linked to the coronavirus specific DCS.

16. The method of claim 13, wherein the nanoparticles are at least 5 nm and maximally 40 nm in diameter

17. The method of claim 13, wherein the method further comprises after step b) a wash step.

18. The method of claim 13, wherein the detection molecule comprises a detectable tag bound to the DCS and a second detectable agent capable of binding and/or detecting the detectable tag when complexed, the method further comprising after step b) contacting the reaction matrix with a solution comprising the second detectable agent, and optionally subsequently contacting the reaction matrix was a wash solution.

19. The method of claim 18, wherein the detectable tag is biotin and the second detectable agent is streptavidin linked to nanoparticles.

20. The method of claim 14, wherein the the DCS is an aptamer specific to SARS-CoV-2.

21. The method of claim 20, wherein the aptamer comprises a nucleic acid sequence selected from SEQ ID NO:1-13.

22. The method of claim 14, wherein step (c) comprises visualizing the detectable complex on the matrix in less than a minute from contacting the matrix with the sample.

23. A method of detecting coronavirus in a sample, the method comprising:
a) contacting a sample with a reaction matrix within a gravitational flow through assay, the gravitational flow through assay comprising:
(i) a cassette comprising an absorbent pad;
(ii) a reaction matrix comprising nitrocellulose layered on top of the absorbent pad within the cassette, the reaction matrix complexed with a coronavirus-specific DNA capture sequence (DCS); and
b) allowing the sample to flow through the reaction matrix by gravity;
c) detecting the presence of a complex of the coronavirus-specific DCS and a coronavirus nucleic acid sequence from the sample, wherein detection of the complex indicates the presence of coronavirus in the sample.

24. The method of claim 23, wherein the DCS are selected from SEQ ID NO:1-13.

25. A method of detecting coronavirus in a sample, the method comprising:
a) contacting a sample with a reaction matrix within a gravitational flow through assay, the gravitational flow through assay comprising:
(i) a cassette comprising an absorbent pad;
(ii) a reaction matrix comprising nitrocellulose layered on top of the absorbent pad within the cassette, the reaction matrix complexed with a coronavirus-specific DNA capture sequence (DCS) linked to a detection tag; and
b) allowing the sample to flow through the reaction matrix by gravity;
c) contacting the reaction matrix with a solution comprising a second detectable agent;
d) washing the reaction matrix with a solution; and
e) detecting the presence of a complex of the coronavirus-specific DCS, detection tag, second detectable agent and a nucleic acid sequence from the sample, wherein detection of the complex indicates the presence of coronavirus in the sample.

26. The method of claim 25, wherein step (e) comprises visualizing a change in color on the reaction matrix when the complex is detected.

27. A method of making a gravity flow through assay for coronavirus, comprising:
a) conjugating a coronavirus-specific DNA capture sequence (DCS) with a detection tag;
b) contacting the coronavirus-specific DCS with a reaction matrix comprising nitrocellulose;
c) exposing the reaction matrix to UV light for a sufficient time to link the coronavirus-specific DCS with the reaction matrix;
d) layering in a cassette (i) an absorbent pad, (ii) the reaction matrix comprising the DCS linked with a detection tag.

28. A kit comprising a gravity flow through assay for coronavirus comprising
(a) an assay comprising
(i) a cassette containing an absorbent pad;
(ii) a porous reaction matrix layered on top of the absorbent pad within the cassette, the reaction matrix comprising nitrocellulose; and
(iii) a detection agent comprising (i) a coronavirus-specific DNA capture sequence (DCS) linked to the matrix;
(b) a solution comprising a detectable molecule;
(c) a wash solution and
(d) instructions for use.

29. The kit of claim 28, wherein the DCS comprise one or more polynucleotides selected from SEQ ID NO:1-13.
